(19) United States
(12) Patent Application Publication
(10) Pub. No.: US 2012/0115177 A1
(43) Pub. Date: May 10, 2012

(54) Compositions and Methods for Diagnosis of Shock

(75) Inventor: John Rodenrys, La Jolla, CA (US)

(73) Assignee: ANAZYME, LLC, La Jolla, CA (US)

(21) Appl. No.: 13/146,689

(22) PCT Filed: Aug. 5, 2009
(86) PCT No.: PCT/US09/52784

§ 371 Date: Jan. 11, 2012

Related U.S. Application Data
(63) Continuation-in-part of application No. 12/360,976, filed on Jan. 28, 2009.
(60) Provisional application No. 61/024,997, filed on Jan. 31, 2008.
(60) Provisional application No. 61/211,832, filed on Apr. 2, 2009.

(51) Int. Cl. G01N 21/75; C12Q 1/34; C12Q 1/02; C12Q 1/37

(52) U.S. Cl. 435/23; 435/18; 436/111; 435/29; 436/128; 436/119; 436/120; 436/129; 436/93; 436/98

(57) ABSTRACT

Methods and kits for diagnosis and staging of shock, and especially non-septic shock are presented in which protease activities and/or volatile compounds are measured from a biological sample to so identify and/or stage shock.

This application claims priority to our copending U.S. provisional application with the Ser. No. 61/211,832, which was filed Apr. 2, 2009, and further claims priority to our U.S. application with the Ser. No. 12/360,976, which was filed Jan. 28, 2009.

### FIELD OF THE INVENTION

The field of the invention is methods and compositions for diagnosis and/or staging of various conditions, and especially of various shock conditions.

### BACKGROUND OF THE INVENTION

Matrix metalloproteinases (MMPs) and selected other proteases are known to degrade numerous substrates, and especially extracellular matrix proteins. More recently, it was also discovered that some MMPs specifically process certain cell surface receptors to so modify the receptor function, while other MMPs are involved in the generation of apoptotic ligands and chemokine modulation. Not surprisingly, MMPs are therefore involved in various physiological processes, including cell proliferation, migration (adhesion/dispersion), differentiation, angiogenesis, apoptosis and host defense.

Based on the relatively diverse role of MMPs, various diseases have been correlated with MMP activity. For example, increased matrix metalloproteinase-2 (MMP2) transcription has been associated with impaired adipogenesis in type 2 diabetes mellitus (Biochem Biophys Res Commun. 2008 Jan. 5), and circulating levels of matrix metalloproteinase (MMP)-10 were reported to be related to inflammation (J Thromb Haemost. 2007 January; 5(1):91-7). In other examples, the kallikrein-kinin system was shown to be significantly implicated in numerous conditions, including inflammation, cancer, and in certain pathologies related to cardiovascular, renal and central nervous systems. In still further examples, diabetes was shown to be associated with increased MMP2 expression as disclosed in U.S. Pat. App. No. 2007/218519, and hypertension was associated with altered kallikrenin activities as described in EP00234095A. Consequently, where exacerbated MMP activity is associated with a disease, various forms of treatment of such diseases with MMP inhibitors were proposed as described, for example, in U.S. Pat. App. No. 2007/294107.

Non-septic shock can have various etiologies (hypovolaemic, cardiogenic, distributive, obstructive, endocrine, etc.) and is often diagnosed by overall clinical appearance such as skin tone, blood pressure, heart rate, oxygenation level, mental clarity, etc. However, and especially with compensated non-septic shock, accurate diagnosis is often difficult, and metabolic analysis may assist in the clinical finding. For example, lactic acid may be used as a parameter. More recently, secretory phospholipase A2 (sPLA2), procalcitonin (PCT) and C-reactive protein (CRP) levels were reported as analytical tools for diagnosis and differentiation of septic shock and non-septic shock (Critical Care 2000, 4(Suppl 1):P68). Similarly, diagnosis of septic shock and SIRS has been performed using a multi-marker analysis as described in U.S. Pat. App. No. 2005/164238. However, such analyses are often time consuming, relatively expensive, and can often not be carried out at the point of care (e.g., accident site).

Therefore, while numerous methods of diagnosing shock art (and especially non-septic shock) are known in the art, all or almost all of them suffer from various disadvantages. Thus, there is still a need to provide improved diagnostic tools and methods for identification and staging of non-septic shock.

### SUMMARY OF THE INVENTION

The present invention is directed to compositions and methods of diagnosing and/or staging shock, and especially non-septic shock, in which the amount of one or more serum components and/or volatile components are used to identify and/or stage shock.

In one especially contemplated aspect of the inventive subject matter, a method of diagnosing a shock condition includes a step in which a patient sample is analyzed by measuring the activity of at least one protease in the sample and/or by measuring the amount of at least one volatile compound in the sample to so obtain a test result (that is based on the activity and/or quantity). The so obtained test result is then correlated with at least one of presence and progression of the shock condition in the patient, typically using reference standards from healthy individuals.

For example, where the analysis is based on the activity of at least one protease, it is especially preferred that the activity is measured by measuring a decrease in a serum protein, an increase in a protease cleavage product in serum, and/or by monitoring the cleavage of a labeled protease substrate. Most typically, the protease is a serine protease (e.g., thrombin, plasmin, trypsin, and kallikrein), or one or more matrix metalloproteinases. In especially contemplated methods, a step of determining a ratio of at least two protease activities is included where the ratio or activities is correlated with the presence and/or progression of (non-septic) shock in the patient.

On the other hand, where the analysis is based on the quantity of one or more volatile compounds, it is especially preferred that the volatile compounds are volatile amines, volatile aldehydes, and/or volatile sulfur-containing compounds, which are typically measured from whole blood, serum, or breath. Most preferably, such volatile compounds include putrescine, cadaverine, methional, pentanoic acid, 2-furfurylthiol, citronellal, (E)-2-octenal, 2-nonenal, phenylactealdehyde, 2,6-dimethyl-5-heptanal, 2,3-diethyl-5-methylpyrazine, and 4-acetylmethylcyclohexene.

Various objects, features, aspects and advantages of the present invention will become more apparent from the following detailed description of preferred embodiments of the invention.

### BRIEF DESCRIPTION OF THE DRAWING

FIG. 1 is a graph illustrating protease activity of an exemplary protease in serum of animals subjected to hypovolemic shock and control animals post resuscitation.

FIG. 2A is an exemplary HPLC trace of serum proteins from control animals.

FIG. 2B is an exemplary HPLC trace of serum proteins from animals subjected to hypovolemic shock.

### DETAILED DESCRIPTION

According to the present invention, various pathological conditions are correlated with the presence or absence of certain compounds in a patient sample to so arrive at a diagnosis or staging of the condition. In especially preferred aspects, compounds that are correlated with the pathological conditions include proteases, their substrates and/or reaction products, and various volatile compounds, where the pathological conditions include shock, and especially non-septic shock, various bowel diseases (and particularly inflammatory bowel disease), and gastrointestinal diseases in premature babies or infants, and even autism.

Thus, particularly preferred methods include diagnostic methods in which a condition (e.g., shock, non-septic shock, bowel disease, etc.) is ascertained or staged by obtaining a patient sample. The sample is then analyzed by qualitative, and more typically quantitative analysis of one or more protease activities and/or by qualitative, and more typically quantitative analysis of one or more volatile compounds in the sample to so obtain a test result that is based on the protease activity and/or quantity of volatile compounds. The test result is then correlated with the presence and/or progression of the condition in the patient.

For example, in especially preferred aspects of the inventive subject matter, shock (and especially non-septic shock) is diagnosed, the effectiveness of drug treatment for (non-septic) shock is monitored, and/or prognosis for a patient suffering from (non-septic) shock is provided by measuring protease activity of one or more selected proteases in serum or other body fluid and by correlating the test result with a diagnostic finding, treatment efficacy, and/or prognosis. Most preferably, the activity and/or quantity of at least two proteases are measured in serum, and it is particularly preferred that the proteases are selected from the group of serine proteases and metalloproteinases. Alternatively, or additionally, further diagnostic markers may be used in correlation or without correlation to measured protease activity.

While suitable patient samples may include numerous body fluids (e.g., whole blood, plasma, serum, sweat, saliva, tear fluid, spinal fluid, urine, etc.), tissue samples (e.g., obtained via biopsy, skin peels, or other physical removal from the body), and even breath, it is especially preferred that blood is drawn from a mammal (typically human or vertebrate) and that the cellular fraction is then removed to obtain the plasma or serum fraction (typically via centrifugation).

Protease activity is then measured by aliquoting small samples to a multiwell plate or other carrier together with appropriately labeled substrates that are most preferably specific for the selected protease (e.g. metallo-, serine, acid and sulfhydryl proteases, cysteine (thiol) proteinases, aspartic proteinases). In particularly preferred aspects, the protease activity is determined for at least one, and most typically at least two of thrombin, plasmin, trypsin, kallikrein, and selected MMPs (e.g., various collagenases, gelatinases, stromelysin-type, and/or membrane-type). The so obtained test result is then compared to corresponding test results from healthy individuals, and the particular activity profile is then associated with a diagnostic finding, treatment efficacy, and/or prognosis.

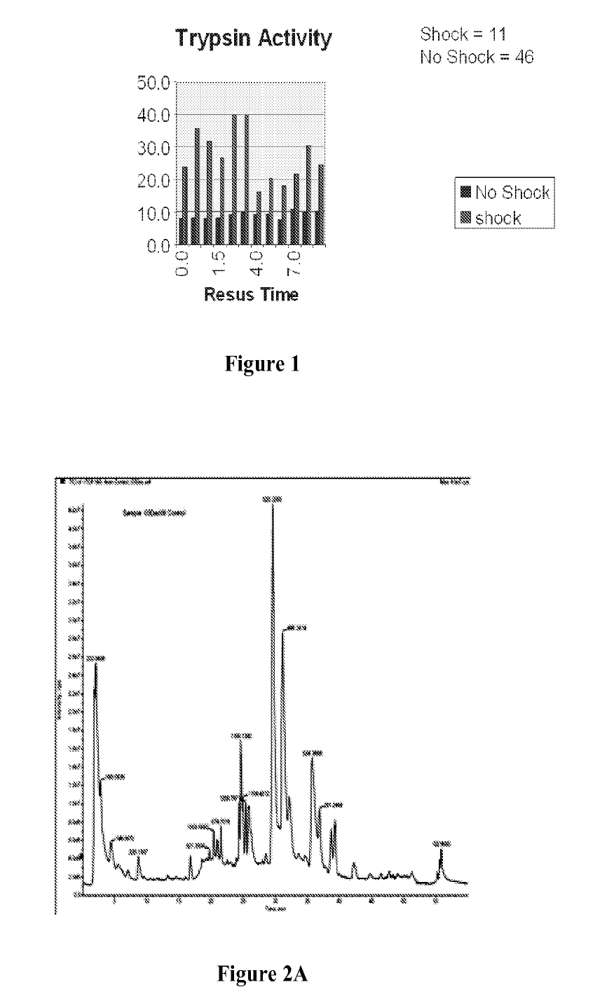

FIG. 1 illustrates an exemplary test result from an experiment in which the trypsin activity from porcine serum was measured. Here, one group was subjected to hypovolumetric shock (shock) and the other group was not subjected to hypovolumetric shock (no shock). As can be clearly seen, trypsin activity was significantly and consistently higher in the animals suffering from non-septic shock, typically at a 2-3 fold higher rate. In this exemplary graph, the resuscitation time is in hours and trypsin activity is in units/ml. Of course, it should be appreciated that numerous alternative proteases could also be monitored based on protocols well known in the art. Moreover, it should be appreciated that more than one protease activity as well as also lipase activity will be measured to obtain additional clinically relevant data.

While it is generally preferred that protease activity is measured from at least two distinct proteases in the same serum at substantially the same time, it should be appreciated that only one protease activity may be measured. However, in most preferred aspects, at least two, three, four, and even more protease activities may be measured. Typically, multiple measurements will be performed serially or in parallel in single test device. Thus the activity determinations may be separated in a spatial, spectroscopic, or temporal fashion. Where appropriate, deconvolution may be needed to provide individual results. Alternatively, combined results may also be indicative. Moreover, and especially where multiple data points for a single enzyme are measured, kinetic data (e.g., slope of activity over time) may be used in generation of test results. Therefore, the test results may be in the form of numeric output (e.g., single test value), graphic output (e.g., linear or sigmoidal graph), calculated output (e.g., slope of graph), or ratio of any of the above for multiple measurements of a single protease or of multiple proteases.

Protease tests will typically be based on currently known methods and materials, and it is especially preferred that such tests will include labeled substrates with high specificity to the respective protease. For example, suitable thrombin substrates include commercially available AMC-labeled synthetic peptides (e.g., Technothrombin TGA Fluorogenic substrate [Z-G-G-R-AMC], commercially available from DiaPharma). Similarly, contemplated trypsin substrates include labeled aromatic amides of N-alpha-protected arginine that produce an aromatic amine upon hydrolysis (e.g., BAPNA releases p-nitroaniline, and BANA releases 2-amino-naphthalene that is detected by diazotization and coupling with N-(1-naphthyl)-ethylenediamine to form an azo-dye). Further alternative trypsin substrates include those of U.S. Pat. No. 6,770,764, which is incorporated by reference herein.

Suitable kallikrein substrates include Chromogenix-S2302 (e.g., H-D-Prolyl-L-phenylalanyl-Larginine-p-nitroaniline dihydrochloride commercially available from DiaPhama), and plasmin substrates include Chromogenix-S2403 (e.g., L-Pyroglutamyl-L-Phenylalanyl-L-Lysine-p-Nitroaniline hydrochloride).

Likewise, there are numerous MMP substrates known in the art and all of the known substrates are deemed suitable for use herein. For example, contemplated MMP-2 substrates are synthetic peptides as described by (e.g., Murphy, G., et al. 1994. J. Biol. Chem. 269, 6632). Other MMP substrates can be obtained from various commercial sources, including Anaspec (San Jose, Calif. 95131), and Biomol International (Plymouth Meeting, Pa. 19462).

Similarly, there are numerous lipase substrates known in the art, and all of the currently known lipase substrates and methods are deemed suitable for use herein. For example, lipase activity can be determined using alkaline titration of fatty acid liberated from triglyceride gum arabic emulsions, measurements of the decrease in turbidity of a triglyceride (olive oil) emulsion, and various colorimetric method using a synthetic substrate containing thiol ester of a short chain acid, or measurements of fluorescent phospholipid substrates (see e.g., Chembiochem. 2007 Sep. 3; 8(13):1555-69, or J Lipid Res. 2007 February; 48(2):472-82). Further suitable methods and compounds are described in “Lipase and phospholipase protocols” by Mark Henry Doolittle, Karen Reue, Humana Press, ISBN: 0896035468, which is incorporated by reference herein.

Consequently, it should be appreciated that the label in the protease or lipase substrate may vary and the particular label will depend at least to some degree on the specific protease and desired assay type/condition. Analytic methods for protease activity will therefore include fluorescence, luminescence, UV/VIS absorption, radiometric methods, etc. Especially preferred methods will also include those in which the test result can be determined using the unaided eye (e.g., using colloidal gold labeled antibody, or using one or more chromogenic dyes). Alternatively, non-labeled substrates may be also used and detection is then preferably performed using separation methods and most preferably using various forms of mass spectroscopy (e.g., MALDI-TOF, ES-MS, etc.) and/or chromatographic methods (e.g., HPLC, FPLC, etc). Additionally, it is generally preferred that the analytic method will allow for multiplexed analysis of at least two proteases.

In yet further contemplated aspects, protease activity may also be determined by quantification of the proteases present in the biological sample, or indirectly measured (by quantification of expression of proteases) using proteomic (by antibody arrays) and genetic methods, and particularly genetic array technologies that allow for quantification of expression levels. For example, expression levels may be monitored using rtPCR of mRNA or hnRNA, most typically with whole blood as source material.

Moreover, it should be noted that protease assays need not be limited to detection of synthetic and/or labeled substrates and/or the protease enzymes, but that contemplated assays also include tests in which protease substrates and/or digestion products are measured from the patient sample. For example, contemplated tests may include those in which numerous fragments of cleaved cellular receptors are detected and/or quantified. For example, suitable digestion products include insulin receptor alpha, CD18 receptor, leptin receptors, VEGF receptors, etc. as well as digestive fragments of antibodies and/or soluble or membrane-bound proteins.

Regardless of the manner of obtaining protease activities, it should be appreciated that the results may be individually used, or may be used to form a combined protease activity index, and/or may be used to determine a ratio of one or more first protease activities against one or more distinct second protease activities. Such ratios may be specific for determination of a stage in shock, for progression, and/or be indicative of treatment success or failure. Most typically, and compared against a control value from a healthy individual, higher or increasing protease activities will generally be indicative of presence of deepening non-septic shock while lower or declining protease activities will generally be indicative of absence of non-septic shock or effective treatment.

Therefore, it should be recognized that contemplated test results for protease or lipase activity may be used as diagnostic tool to confirm or indicate the diagnosis of non-septic shock. For example, where the measured protease activities or activity profiles are above a threshold level considered normal for a healthy person, diagnosis of non-septic shock may be confirmed. Similarly, and especially where multiple measurements of protease activity are made over time, decline or incline in activity or change in ratio may be correlated with treatment success, progression of non-septic shock (e.g., compensated to decompensated). Likewise, measurements may be correlated with expected mortality or other prognosis.

In still further contemplated aspects, cellular components other than proteases may also be measured, and particularly contemplated components include intra and extracellular enzymes, membrane-bound enzymes, structural proteins, cytokines, and various messenger substances (e.g., cytokines, chemokines, etc.). Such cellular components may therefore replace measurement of one or more proteases, or may be used in addition to measurement of the proteases. For example, where the cellular component is an enzyme, suitable enzymes will especially include various hydrolases (e.g., phosphatases, lipases, etc.), kinases (and especially those involved in cell signal transduction such as G-protein coupled kinases, tyrosine kinases, etc.), and enzymes associated with energy metabolism and particularly anaerobic energy metabolism. Contemplated structural proteins will especially include collagens and fragments thereof, while contemplated cytokines will include pro-inflammatory cytokines. Particularly contemplated messenger substances will include chemokines and hormones.

Especially contemplated cellular non-protease components that have been identified as markers of non-septic shock include fatty acid binding protein (and all fragments and/or isoforms or family members, including FABP1-FABP11, and FABP5-like 1-7), neuropeptide Y (NPY), and various members of the mucin protein family (MUC1 through MUC20). In still further unexpected results, the inventor found increased concentration of hemoglobin in the samples. Therefore, free hemoglobin and myoglobin may also serve as a potential marker. Moreover, the inventors also discovered in in vivo model systems of hypovolaemic shock, discoloration of digits and nails (most typically redness due to increased hemoglobin) and discoloration of peripheral skin regions (e.g. tip of scotum).

Additional non-protease components have been identified as markers of non-septic shock and include several degradation products from the intestine (e.g., mucins as already noted above, or intestine specific free fatty acid protein), several degradation products from the pancreas (including digestive enzymes themselves after degradation), various lipid fragments (e.g., free fatty acids), numerous plasma protein degradation fragments, and endothelial fragments derived from the endothelial membrane (e.g., extracellular domains of insulin receptor fragments, VGFR2 fragments, ICAM-1, beta 2 adrenergic receptor, glycocalyx protein fragments, etc.).

Additionally, it was also discovered that further systemically observable markers can be indicative of various pathological conditions, and especially non-septic shock. Among other compounds, especially contemplated markers include olfactorially perceptible markers, which are most likely due to emission of volatile organic compounds that produced a characteristic odor. Thus, it should be appreciated that the presence, production, and/or release of various volatile compounds is associated with certain conditions and diseases, and especially shock (septic and non-septic). In especially contemplated aspects, the volatile compounds are odorous compounds and present in whole blood, serum, plasma, breath, urine, in peritoneal and other body fluids, and/or are exuded through the skin. While not limiting to the inventive subject matter, it is contemplated that the volatile compounds may include thiol compounds (in oxidized or reduced form), volatile amines, volatile carboxylic acids, and volatile aldehydes or alcohols.

While not wishing to be bound by any specific theory or hypothesis, it is generally contemplated that the volatile compounds are generated by enzymatic reactions of selected proteases (e.g., matrix metalloproteinases, thrombin, plasmin, trypsin, chymotrypsin, elastase and kallikrein), various lipases (e.g., extracellular lipases such as gastric lipases, pancreatic lipases), certain amylases (e.g., salivary or pancreatic alpha-amylases) and reactions oxygen radicals. Therefore, it should be appreciated that detection of abnormal levels of volatile compounds, and especially odorous volatile compounds may be useful for detection and diagnosis of a disease that is caused by and/or associated with abnormal (and typically increased) protease, lipase, amylase activity or oxygen free radical activity.

In one exemplary test, one group of rats was treated (subjected to three forms of shock, hypovolumetric, endotoxic, and septic shock, respectively) and the other group was kept as control and not subjected to shock. Animals that were subjected to hypovolumetric, endotoxic, or septic shock exhibited a significant and distinct (sulfurous, cadaveric) malodor that indicated presence of volatile compounds (likely volatile thiols and/or hydrogen sulfide, and cadeverine), while the animals of the control group did not exhibit unusual odor.

Therefore, the inventors also contemplate methods for detection and/or diagnosis of a disease that is caused by and/or associated with abnormal (and typically increased) protease, lipase, or amylase activity in which presence of a volatile compound is detected and/or quantified. The so obtained test result is then correlated with a diagnosis or even prognosis relative to the disease, and most preferably (non-septic) shock. In especially preferred aspects, the detection is performed using direct spectroscopic methods or via a chemical reaction in which the volatile compound reacts in a reaction that produces a measurable product (e.g., UV/VIS spectroscopically product of precipitate). In further especially preferred aspects, the volatile compound is measured in the breath of a patient, but may also be measured on the skin or in fluids that have contacted the breath or the skin of the patient (e.g., to concentrate the volatile compound). The type and/or quantity of the volatile compound is then correlated with a disease or condition.

Therefore, according to the present inventive subject matter, shock (e.g., septic or non-septic) is diagnosed, the effectiveness of drug treatment for shock is monitored, and/or prognosis for a patient suffering from shock is provided by measuring the type and quantity of a volatile compound that emanates from a patient. In one especially preferred aspect of the inventive subject matter, breath of a mammal (e.g., rodent, canine, human) is directly analyzed using a gas analysis device (e.g., IR spectrometer) to detect the volatile compound. The so obtained test result is then compared to corresponding test results from healthy mammals, and the particular chemical profile is associated with a diagnostic finding, treatment efficacy, and/or prognosis.

With respect to the volatile compound it is contemplated that the compound is generated by the action of at least one of a protease, lipase, amylase upon a substrate that is present in the mammal under normal and pathological conditions, wherein the substrate concentration may be elevated under pathological conditions to so contribute to the abnormal levels of the volatile compound and/or wherein the amount (and/or activity) of the protease, lipase, and/or amylase may be elevated under pathological conditions to so contribute to the abnormal levels of the volatile compound. Alternatively, it is contemplated that the volatile compound may also be liberated in a secondary reaction from a degradation product that is produced by the protease, lipase, and/or amylase (e.g., via action of a dehydrogenase, esterase, oxidoreductase, oxygen free radical, metabolic enzymes like ornithine decarboxylase, etc.).

Therefore, it should be appreciated that the nature of the volatile compound may vary significantly. However, particularly contemplated compounds will be odorous compound, and especially odorous compounds that are released from enzymatic degradation of serum and/or extracellular proteins. Thus, contemplated volatile compounds include hydrogen sulfide, methyl (or alkyl)mercaptan, acetaldehyde, propionaldehyde, butyraldehyde, valeraldehyde, butyric acid, caproic acid, putrescine, spermidine, ornithine, and amino acid conjugates thereof. Further detected volatile compounds include volatile amines, volatile aldehydes, and/or volatile sulfur-containing compounds, which are typically measured from whole blood, serum, or breath. Most preferably, such volatile compounds include putrescine, cadaverine, methional, pentanoic acid, 2-furfurylthiol, citronellal, (E)-2-octenal, 2-nonenal, phenylactealdehyde, 2,6-dimethyl-5-heptanal, 2,3-diethyl-5-methylpyrazine, and 4-acetylmethylcyclohexene. Moreover, it should be appreciated that these compounds may also be found in various combinations, which may form (together with other gases, and especially carbon dioxide, or by themselves) a specific signature that is diagnostic for the disease or condition. As used herein, the term “volatile” in conjunction with a compound associated with the pathological condition refers to an organic compound with a relatively small molecular weight (typically Mw of less than 500, more typically less than 300, and most typically less than 200), which will typically include at least one non-carbon and non-hydrogen heteroatom (e.g., O, N, or S). Volatile compounds will further typically exhibit a characteristic and perceptible odor when present in aqueous solution at 20° C. and at a concentration of less than 1 mM.

Consequently, it should be recognized that the detection of contemplated compounds may vary considerably. However, in especially preferred aspects, the detection is a direct detection method in which breath, sweat, or another body fluid is analyzed for the type and quantity of the volatile compound. For example, suitable detection methods include gas analysis using IR, UV, and/or VIS spectroscopy in which absorption is directly correlated with the concentration of the compound. Alternatively, the volatile compound may also react with a chromogenic substrate or enzyme system that produces a dye. Where desirable, the volatile compound may be temporarily adsorbed to a carrier or absorbed in a solvent from breath to so allow for concentration of the volatile compound. Moreover, such adsorption or absorption also advantageously allows for temporary storage and transport of the compound to a remote (relative to the patient) analytic device. Therefore, suitable analytic devices also include gas chromatographs and liquid chromatographic devices, which may or may not be coupled to further analytic devices (e.g., mass spectrometer). Detection and quantification in such analytic devices is then performed using methods well known in the art.

Alternatively, and especially wherein the volatile compound is not present in breath, or exuded in larger quantities from the skin (and most typically apocrine and eccrine glands) it is contemplated that the volatile compounds may be adsorbed on a solid carrier or absorbed by a suitable solvent for concentration and further analysis.

Depending on the particular type of volatile compound and manner of detection, it should be appreciated that the detection may not only be direct, using spectroscopic methods, but also indirect, using secondary reactions that produce a specific and quantifiable product. For example, where the volatile compound is a thiol, visually detectable reagents such as 5,5′-dithiobis-(2-nitrobenzoic acid (DTNB) or fluorescence detectable reagents such monobromobimane can be used. On the other hand, where the volatile compound is an amine, an amine oxidase may be employed in the generation of a quantifiable signal.

Thus, collection of the volatile compounds may be from various sources, and most preferably from, blood, breath, and/or exudate from skin. However, in further alternative aspects, various biological fluids may be obtained from the patient (e.g., saliva, blood, serum, urine, etc.) that may serve as the source for the volatile compounds. As noted above, the collection may be in-line with the quantification, or may be performed for transport, storage, and/or concentration of the volatile compound.

Once identified and/or quantified, it is noted that the volatile compound per se, or relative quantities to at least one other compound may be used to establish a diagnostic finding. For example, the quantitative ratio of two or more thiol compounds or a thiol compound and a short chain carboxylic acid and/or aldehyde may be used to determine status of the patient. Of course, it should be appreciated that individual concentrations or ratios measured from healthy subjects will be used to establish a control against which the patient in question is measured.

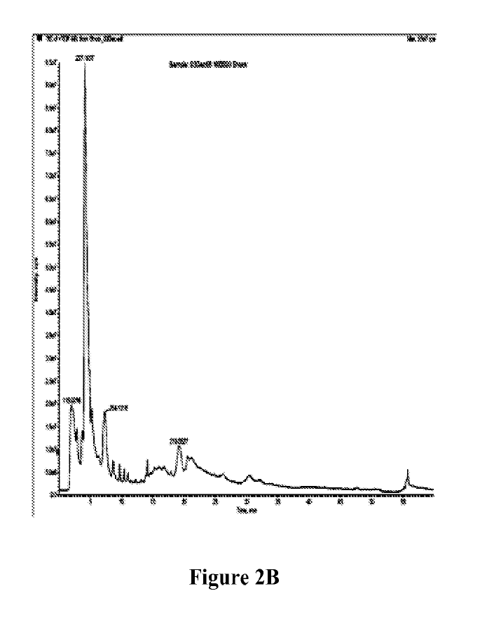

In still further contemplated aspects, shock or other pathological conditions may be determined and/or staged using various markers as apparent from serum profile. For example, the inventors noted that not only presence of certain markers (volatile markers and non-volatile markers), but also absence or decreased quantities of one or more markers can be employed to determine if a shock condition (septic and/or non-septic) is present. For example, in an experiment using rats, blood was collected pre-shock and post-shock, and analyzed via mass spectroscopy. Here, the plasma of rats subjected to hypovolumetric shock showed by mass spectrometric analysis an extensive destruction of characteristic organic compounds when compared to plasma of the same animals before shock. There are also lower molecular weight organic fragments generated during shock that are not present in control plasma. As can be taken from FIGS. 2A (pre-shock) and 2B (post-shock), the difference in analytes was significant and demonstrated an absence and/or reduction of certain molecules after shock. Consequently, it is contemplated that an assay to determine shock may include a step in which the presence and/or quantity of one or more analytes is determined, and shock is confirmed if those analytes are no longer present or detectable with certain conventional methods (e.g. by antibody binding) and/or reduced in quantity.

Consequently, contemplated kits for diagnosis of non-septic shock will include one, and more preferably two or more protease substrates (most preferably labeled) and an instruction to measure protease activity in a patient sample to ascertain or monitor non-septic shock. Most typically, such kit will also include interpretive information that provides a user with protease activities expected to be within a range considered normal, and protease activities considered to be indicative of non-septic (John, could also be the case in septic shock) shock. Where suitable, such information may also provide further information on ratios of protease activities and associated conditions therewith.

Thus, specific embodiments and applications of proteases and volatile compounds in the diagnosis of shock, including septic and non-septic shock, have been disclosed. It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of diagnosing a shock condition, comprising the steps of:
analyzing a patient sample by measuring at least one of
(a) an activity of at least one protease or lipase in the sample, and
(b) a quantity of at least one volatile compound in the sample, to so obtain a test result based on the at least one of the activity and quantity; and
correlating the test result with at least one of presence and progression of the shock condition in the patient.

2. The method of claim 1 wherein the test result is based on the activity of at least one protease or lipase.

3. The method of claim 2 wherein the activity is measured by measuring a decrease in a serum protein or by measuring an increase in a protease or lipase cleavage product in serum.

4. The method of claim 2 wherein the activity is measured by monitoring cleavage of a labeled protease or lipase substrate.

5. The method of claim 2 wherein at least two protease or lipase activities are measured.

6. The method of claim 2 wherein the protease activity is a serine protease.

7. The method of claim 6 wherein the serine protease is selected from the group consisting of thrombin, plasmin, trypsin, and kallikrein.

8. The method of claim 5 wherein one of the at least two proteases is a matrix metalloproteinase.

9. The method of claim 5 further comprising a step of determining a ratio of the at least two protease activities and correlating the ratio with the at least one of presence and progression of septic and non-septic shock in the patient.

10. The method of claim 1 wherein the test result is based on the quantity of at least one volatile compound.

11. The method of claim 10 wherein at least two volatile compounds are measured.

12. The method of claim 10 wherein the volatile compound is a volatile amine.

13. The method of claim 10 wherein the volatile compound is a volatile aldehyde.

14. The method of claim 10 wherein the volatile compound is a volatile sulfur-containing compound.

15. The method of claim 10 wherein the volatile compound is selected from the group consisting of putrescine, cadaverine, methional, pentanoic acid, 2-furfurylthiol, phenylactealdehyde, 2,6-dimethyl-5-heptanal, (E)-2-octenal, 2-nonenal, citronellal, 2,3-diethyl-5-methylpyrazine, and 4-acetylmethylcyclohexene.

16. The method of claim 10 wherein the volatile compound is measured from whole blood, serum, or breath.
